(19) United States
(12) Patent Application Publication
(10) Pub. No.: US 2015/0091592 A1
(43) Pub. Date: Apr. 2, 2015

(54) TEST STRIP RESISTANCE CHECK

(71) Applicant: Cilag GmbH International, Zug (CH)

(72) Inventor: David ELDER, Inverness (GB)

(73) Assignee: Cilag GmbH International, Zug (CH)

(21) Appl. No.: 14/042,299

(22) Filed: Sep. 30, 2013

(51) Int. Cl. G01N 27/04; G01N 33/66

(52) U.S. Cl. CPC G01N 27/04; G01N 33/66
USPC 324/692; 324/693

(57) ABSTRACT

A system for determining usability of an analytical test strip having two electrodes connected in series with a sample cell includes a test meter configured to receive the test strip. The sample cell receives a fluid sample, the sample cell with the received fluid sample having a frequency-dependent impedance. A microprocessor and circuit in the test meter cause application of an AC waveform across the sample cell via the electrodes upon detection of sample in the sample cell and concurrently measure of a current through the electrodes. The AC waveform has a frequency at which the characteristic impedance is substantially zero. The measured current is inversely proportional to a series resistance of the two electrodes. A hand-held test meter and a method for determining usability of an analytical test strip are also described.

### TECHNICAL FIELD

The present invention relates, in general, to the field of analyte measurement and, in particular, to test meters and related methods for proactively detecting error conditions of analytical test strips based on specified criteria.

### DESCRIPTION OF RELATED ART

The determination (e.g., detection or concentration measurement) of an analyte in a fluid sample is of particular interest in the medical field. For example, it can be desirable to determine glucose, ketone bodies, cholesterol, lipoproteins, triglycerides, acetaminophen or HbA1c concentrations in a sample of a bodily fluid such as urine, blood, plasma or interstitial fluid. Such determinations can be achieved using a hand-held test meter in combination with analytical test strips (e.g., electrochemical-based analytical test strips). Analytical test strips generally include a sample cell (also referred to herein as an “analyte chamber”) for maintaining a liquid analyte, e.g., whole blood, in contact with two or more electrodes. Analytes can then be determined electrochemically using signals conveyed by the electrodes.

Since test meters are used to make care decisions relating to medical conditions, it is desirable that these devices measure with as much accuracy and precision as possible. However, the electrodes (or contacts or other electrically conductive components) on the analytical test strip can have defects introduced due to manufacture, storage or handling that that introduce noise or offset during measurement. More specifically, it has been determined that these defects create resistances to measured voltages or currents. It is therefore desirable to measure these effects so they can be be compensated for or to notify a user in advance of obtaining an analyte reading. Moreover, it is desirable to reject test strips with resistances beyond a range in which accuracy can be suitably maintained.

### BRIEF DESCRIPTION OF THE DRAWINGS

Various novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified depiction of a system according to an embodiment of the present invention;

FIG. 2 is an exploded view of an exemplary test strip showing schematic representations of circuit characteristics;

FIG. 3 is a plot of resistance as a function of frequency measured on an exemplary test strip; and

FIG. 4 is a flow diagram depicting stages in an exemplary method for determining usability of an analytical test strip.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, the term “in”, as used throughout this description, does not necessarily require that one component or structure be completely contained within another, unless otherwise indicated.

Throughout the course of discussion, the symbol “j” is used to refer to the imaginary unit, \sqrt{−1}, in conformance with standard notation found in electrical engineering. The symbols “i” or “I” each refer to electric current.

In general, portable test meters, such as hand-held test meters, for use with an analytical test strip in the determination of an analyte (such as glucose) in a bodily fluid sample (i.e., a whole blood sample) according to embodiments of the present invention include a circuit and a microprocessor configured to apply an AC waveform across a sample cell of the test strip and measure the resistance of electrodes that are disposed on the strip while applying the waveform. The AC waveform passes through the capacitance of the sample cell without regard to the DC resistance of the sample cell, permitting the taking of accurate measurements of test-strip resistance.

Hand-held test meters according to embodiments of the present invention are beneficial in that they provide a qualitative determination of test strip usability. For example, the detection of an unusually high resistance can indicate that one or more electrodes on the test strip have been nicked or otherwise damaged or can be indicative of poor manufacture. It is desirable to avoid using such test strips, since the damage to the electrodes may reduce the accuracy of the results.

A problem solved by various embodiments is to accurately measure the resistance of electrodes on the analytical test strip, those electrodes being separated by the sample cell that retains the fluid sample. The DC resistance of the fluid, or of a dry sample cell, can be very high. The electrodes in the sample cell define a capacitor that is electrically in parallel with the DC resistance. Using an AC waveform permits passing current through the capacitor of the sample cell, shorting out the DC resistance.

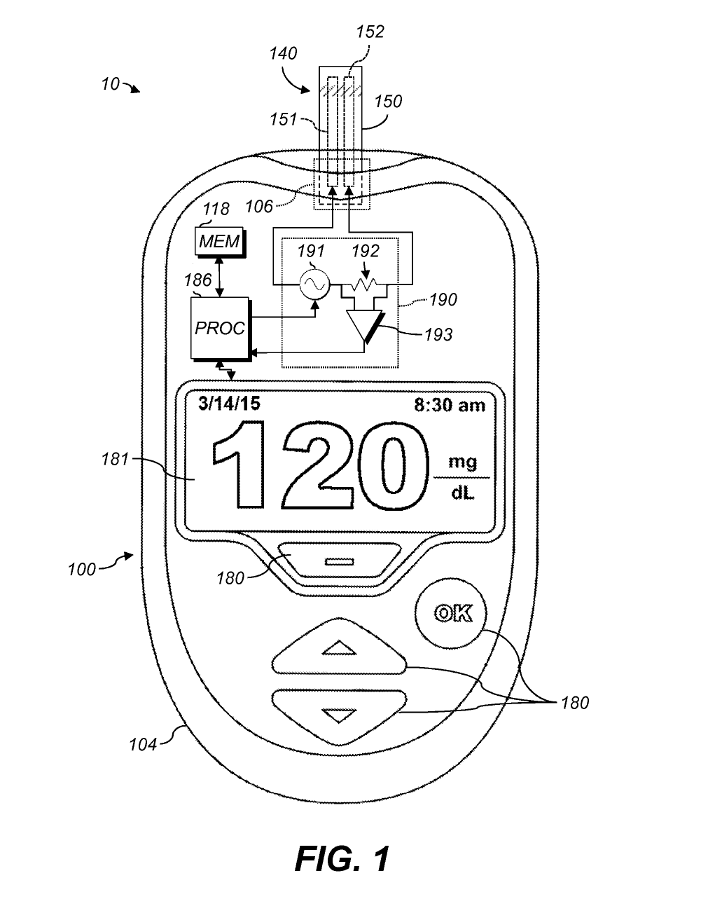

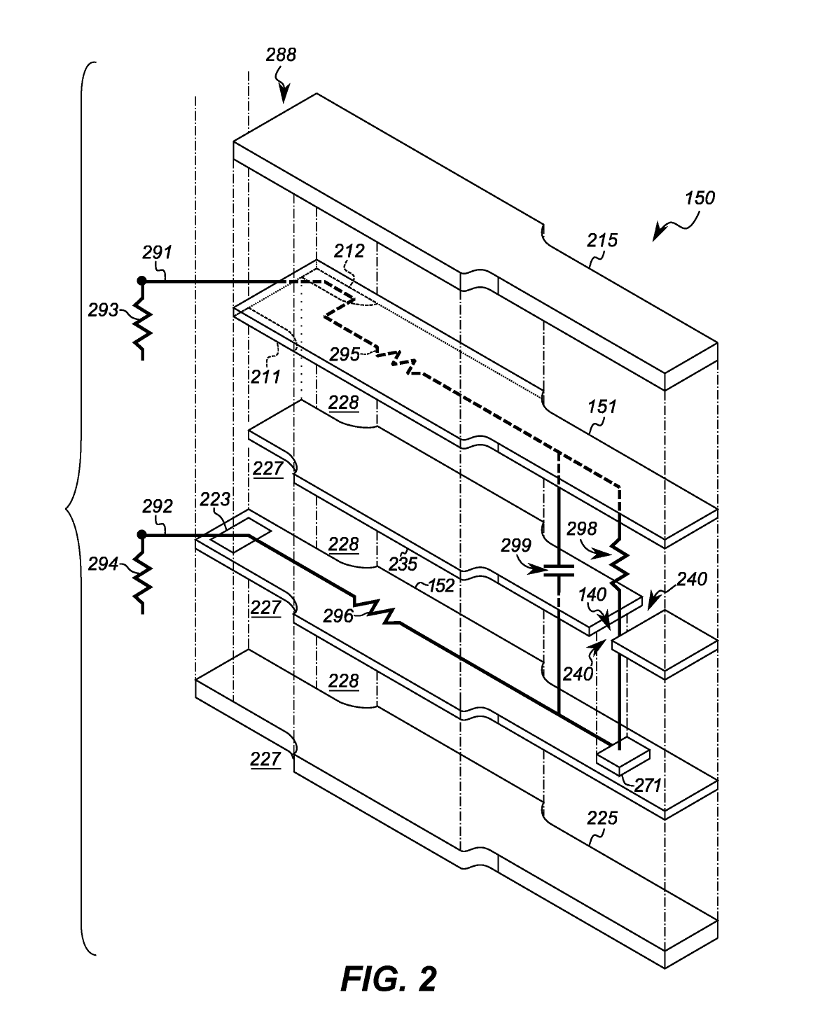

FIG. 1 shows an exemplary system for determining usability of an analytical test strip. The system 10 includes an analytical test strip 150 having two electrodes 151, 152 connected in series with a sample cell 140. The sample cell 140 is adapted to receive a fluid sample and configured so that the sample cell 140 with the received fluid sample has a characteristic impedance that varies with frequency of electrical excitation. This is discussed in greater detail below with reference to FIG. 2.

The herein described system 10 also includes a test meter 100 adapted to receive the analytical test strip 150. The test meter 100 has at least one contained circuit, in this example a resistance-characterization circuit (RCC) 190, and a microprocessor 186. The microprocessor 186 and circuit (the RCC 190) are configured to detect the presence of the fluid sample in the sample cell 140 of the received analytical test strip 150. Based upon the detection of the fluid sample, the microprocessor 186 causes application of an AC waveform across the sample cell 140 via the electrodes 151, 152, and causes concurrent measurement of a current through the electrodes 151, 152, e.g., using a current detector in the RCC 190. The AC waveform has a frequency at which the characteristic impedance is substantially zero, as is discussed below with reference to FIG. 2. The measured current is inversely proportional to a series resistance of the two electrodes 151, 152.

In the example shown, the RCC 190 includes an AC voltage source 191 controlled by the microprocessor 186. The AC voltage source is connected to the electrode 151. A current detector in the RCC 190 includes a resistor 192 in series between the electrode 152 and the AC voltage source 191. The voltage across the resistor 192 is directly proportional to the current through the AC voltage source 191 and the electrodes 151, 152. An amplifier 193 amplifies the voltage across the resistor 192 to provide a voltage signal to the microprocessor 186 that is representative of current through the electrodes 151, 152. In one version, the AC voltage source 191 includes a low-pass filter that receives a square wave from the microprocessor 186 and provides a filtered voltage that is closer to a sinusoid as a result of the filtering. Exemplary low-pass filters for this purpose can include fourth-order filters, multiple feedback low pass filters, and Sallen and Key low pass filters.

The concepts discussed herein can readily be incorporated by one of sufficient skill into a hand-held test meter. One example of a test meter that can be suitably configured is the commercially available OneTouch® Ultra® 2 glucose meter from LifeScan Inc. (Milpitas, Calif.). Additional examples of hand-held test meters that can also be modified are described in U.S. Patent Application Publication Nos. 2007/0084734 (published on Apr. 19, 2007) and 2007/0087397 (published on Apr. 19, 2007) as well as International Publication Number WO2010/049669 (published on May 6, 2010), each of which is hereby incorporated by reference in their entirety.

As noted, the test meter 100 can be a hand-held test meter for use with an analytical test strip 150 in the determination of at least one analyte in a bodily fluid sample. Still referring to FIG. 1, an exemplary test meter 100 can include a housing 104 and a strip port connector (SPC) 106 that is configured to receive the analytical test strip 150, which is inserted into a port of the housing 104. The SPC 106 can include spring contacts arranged so that the test strip 150 can be slid into the SPC 106 to electrically connect the electrodes 151, 152 with the RCC 190. The SPC 106 can also or alternatively include pogo pins, solder bumps, pin or other receptacles, jacks, or other devices for selectively and removably making electrical connections.

Still referring to FIG. 1, the test meter 100 can also include a user interface including, e.g., a display 181 and one or more user interface buttons 180. The display 181 can be, for example, a liquid crystal display or a bi-stable display configured to show a screen image. The exemplary screen image shown in FIG. 1 provides indications of glucose concentration (“120”) and of date and time (“3/14/15 8:30 am”), as well as a units indication (“mg/dL”). The display 181 can also present error messages or instructions to a user on how to perform a test (analyte determination).

The test meter 100 can also include other electronic components (not shown) for applying test voltages or other electrical signals to the analytical test strip 150, and for measuring an electrochemical response (e.g., plurality of test current values) and determining an analyte based on the electrochemical response. To simplify the present descriptions, the figures do not depict all such electronic circuitry.

The RCC 190 can be electrically connected to the sample cell 140 of the received analytical test strip 150 via the strip port connector 106. The RCC 190 can be configured to selectively apply an excitation voltage signal to the sample cell to provide a resultant electrical signal. According to the present invention and as discussed in greater detail in a later portion of this description with reference to FIG. 2, the excitation voltage signal can have an excitation voltage and an excitation frequency greater than a characteristic frequency of the fluid sample.

According to the exemplary embodiment, the microprocessor 186 is disposed within the housing 104. The microprocessor 186 can be adapted to detect the fluid sample in the sample cell 140 and subsequently cause the RCC 190 to apply the excitation voltage signal. For the purposes described herein, the microprocessor 186 can include any suitable microcontroller or micro-processor known to those of skill in the art. One exemplary microcontroller is an MSP430F5138 microcontroller that is commercially available from Texas Instruments, Dallas, Tex. USA. The microprocessor 186 can include, e.g., a field-programmable gate array (FPGA) such as an ALTERA CYCLONE FPGA, a digital signal processor (DSP) such as a Texas Instruments TMS320C6747 DSP, or another suitable processing device adapted to carry out various algorithm(s) as described herein. The microprocessor 186 can include signal-generation and signal-measurement functions, e.g., D/A converters, pulse-train generators, or A/D converters.

In various embodiments, the microprocessor 186 is further adapted to determine whether the resultant electrical signal satisfies a stored resistance criterion. The resistance criterion can be stored, e.g., in a memory block 118. The microprocessor 186 can further be adapted to indicate to a user, via a user interface of the test meter (e.g., a display 181), whether the resultant electrical signal satisfies the stored resistance criterion.

The memory block 118 of the hand-held test meter 100 includes one or more storage device(s), e.g., a code memory (such as random-access memory, RAM, or Flash memory) for storing, e.g., program firmware or software; a data memory (e.g., RAM or fast cache); or a disk (such as a hard drive). Computer program instructions to carry out a suitable algorithm(s) are stored in one of those device(s). The memory block 118 can also or alternatively be incorporated in the microprocessor 186. A Flash or other nonvolatile memory in the memory block 118 can also contain, e.g., graphics to be displayed on the display 181, text messages to be displayed to a user, calibration data, user settings, or algorithm parameters.

The microprocessor 186 can use information stored in the memory block 118 in determining an analyte, e.g., in determining a blood glucose concentration, based on the electrochemical response of analytical test strip. For example, the memory block 118 can store correction tables to adjust the determination of the analyte based on a determined resistance of the test strip 150.

Throughout this description, some embodiments are described in terms that would ordinarily be implemented as software programs. Those skilled in the art will readily recognize that the equivalent of such software can also be constructed in hardware (hard-wired or programmable), firmware, or micro-code. Given the systems and methods as described herein, software or firmware not specifically shown, suggested, or described herein that is useful for implementation of any embodiment is conventional and within the ordinary skill in such arts.

In various embodiments, the strip port connector 106 is configured to operatively interface with the received analytical test strip 150 via the electrodes 151, 152 of the received analytical test strip 150. Each of the electrodes 151, 152 is disposed at least partly in the sample cell 140 of the received analytical test strip 150. The strip port connector 106 can include two electrical contacts (not shown) that electrically connect with the electrodes 151, 152, respectively, when the test strip 150 is inserted into the strip port connector 106, e.g., by a user.

Once the analytical test strip 150 is interfaced with the hand-held test meter 100, or prior thereto, a fluid sample (e.g., a whole blood sample or a control-solution sample) is introduced into the sample cell 140 of the analytical test strip 150. The analytical test strip 150 can include enzymatic reagents that selectively and quantitatively transform an analyte in the fluid sample into another predetermined chemical form. For example, the analytical test strip 150 can be an electrochemical-based analytical test strip configured for the determination of glucose in a whole blood sample. Such a test strip 150 can include an enzymatic reagent with ferricyanide and glucose oxidase so that glucose can be physically transformed into an oxidized form.

FIG. 2 is an exploded view of an exemplary test strip 150, with circuit characteristics represented schematically. Additional details of various exemplary test strips and measurement methods are provided in US Patent Application Publication No. 2007/0074977, incorporated herein by reference in its entirety. The test strip 150 can be, e.g., an electrochemical-based analytical test strip configured for the determination of glucose in a whole blood sample.

A first electrode 151 and a second electrode 152 are arranged to define the sample cell 140. The second electrode 152 is electrically insulated from the first electrode 151, e.g., by an electrically-insulating spacer 235 arranged between the first electrode 151 and the second electrode 152. The sample cell 140 can be formed by removing a portion of the spacer 235, or by disposing two separated portions of the spacer 235 between the first and second electrodes 151, 152. In this example, the electrodes 151, 152 are substantially parallel and electrically isolated, so they can serve as plates of a capacitor, as will be discussed below. In various embodiments, the electrodes 151, 152 can be arranged spaced apart in a facing or opposing faced arrangement, or in other coplanar or non-coplanar configurations.

A first electrically-insulating layer 215, e.g., a top insulator, is disposed over the first electrode 151 and can cover the whole surface or only a portion thereof. A second electrically-insulating layer 225, e.g., a bottom insulator, is disposed beneath the second electrode 152 and can also cover the whole surface or a portion thereof.

The electrodes, e.g., the electrodes 151, 152, can be thin films. In various aspects, electrodes include conductive material formed from materials such as gold, palladium, carbon, silver, platinum, tin oxide, iridium, indium, and combinations thereof (e.g., indium-doped tin oxide or “ITO”). Electrodes can be formed by disposing a conductive material onto the electrically-insulating layers 225, 215 by a sputtering, electroless plating, thermal evaporation, or screen printing process. In an example, the electrode 151 is a sputtered gold electrode disposed over the side not visible in FIG. 2 of the electrically-insulating layer 215, and the electrode 152 is a sputtered palladium electrode disposed over the side visible in FIG. 2 of the electrically-insulating layer 225. Suitable materials that can be employed in the electrically-insulating layers 215, 225 include, for example, plastics (e.g. PET, PETG, polyimide, polycarbonate, polystyrene), silicon, ceramic, glass, and combinations thereof. For example, the first and second insulating layers 215, 225 can be formed from 7 mil polyester substrate(s).

A first electrical contact pad 211 and a second electrical contact pad 212 are electrically connected to the first electrode 151. In this embodiment, each of the contact pads 211, 212 is arranged on the underside of the first electrode 151. The second electrical contact pad 212 is electrically connected to the first electrical contact pad 211. A third electrical contact pad 223 is electrically connected to the second electrode 152 and, according to the depicted version, is applied to a top surface thereof. In various aspects, the contact pads 211, 212, 223 are disposed apart from the first and second electrodes 151, 152 and are in electrical communication therewith. In other aspects, such as depicted according to FIG. 2, the first and second electrodes 151, 152 extend to encompass the pads 211, 212, 223, such that the contact pads 211, 212, 223 are defined areas of the electrodes 151, 152.

In various embodiments, the test meter 100, FIG. 1, can measure the resistance or electrical continuity between two contacts of the SPC 106, FIG. 1, arranged to respectively contact the first electrical contact pad 211 and the second electrical contact pad 212. When the test strip 150 is properly inserted into the test meter 100, the electrical connection between the first and second electrical contact pads 211, 212 shorts the corresponding contacts of the SPC 106. This connection permits the microprocessor 186, FIG. 1, to detect insertion of the test strip 150 and, e.g., wake up from a low-power sleep mode and initiate a fluid-detection cycle. Once a determination is made that the test strip 150 is electrically connected to the test meter 100, the test meter 100 can apply a test potential or current, e.g., a constant current, between the first electrical contact pad 211 and the third electrical contact pad 223. In an example, a constant DC current can be applied into the sample cell 140, and the voltage across the sample cell 140 can be monitored. When the fluid sample has filled the sample cell 140, the voltage across the sample cell 140 will fall below a selected threshold. AC signals, as described herein, can be measured before the sample cell 140 has filled with fluid, or after the sample cell 140 has filled with fluid.

In this exemplary configuration, each of the spacer 235, the second electrically-insulating layer 225, and the second electrode 152 includes corresponding first and second cutout portions 227, 228 provided at a proximal end 288 of the test strip 150. The cutout portions 227, 228 are configured and sized to permit making electrical contact with the first electrode 151, e.g., at the first and second electrical contact pads 211, 212. Also in the illustrated example, the second electrically-insulating layer 225 and the second electrode 152 protrude beyond the spacer 235, the first electrode 151, and the first electrically-insulating layer 215 at the proximal end 288. This configuration permits electrical contact to be made with the second electrode 152, e.g., at the third electrical contact pad 223.

The sample cell 140 includes an aperture 240 arranged so that a fluid sample can be drawn into the sample cell under capillary action. For purposes of this exemplary embodiment, the fluid sample is a whole blood sample and the analyte to be detected is glucose. The capillary action can occur as the fluid sample is brought into contact with edges or sidewalls of the aperture 240. The sample cell 140 can include more than one aperture 240; in the example shown, the sample cell 140 has two laterally-opposed apertures 240. One of the apertures 240 can provide a sample inlet and the other aperture 240 can act as a vent.

In various aspects, the sample cell 140 is adapted for analyzing small-volume samples. For example, the sample cell 140 can have a volume ranging from about 0.1 microliters to about 5 microliters, a volume ranging from about 0.2 microliters to about 3 microliters, or a volume ranging from about 0.3 microliters to about 1 microliter. To accommodate a small sample volume, the electrodes 151 and 152 can be closely spaced in relation to one another. The height of the spacer 235, as shown, defines the distance between the second electrode 152 and the first electrode 151. To provide sample cell volumes in the above ranges, the height of the spacer 235 can be in the range of about 1 micron to about 500 microns, or in the range of between about 10 microns and about 400 microns, or in the range of between about 40 microns and about 200 microns. Further details relating to the construction, design and features of exemplary test strips are given in U.S. Pat. No. 8,163,162, incorporated herein by reference in its entirety.

A reagent layer 271 can be disposed within the sample cell 140 using a process such as slot coating, coating by dispensing liquid from the end of a tube, ink jetting, and screen printing. Such processes are described, for example, in U.S. Pat. Nos. 6,676,995; 6,689,411; 6,749,887; 6,830,934; and 7,291,256; in U.S. Patent Application Publication No. 2004/0120848; and in PCT Application Publication No. WO/1997/018465 and U.S. Pat. No. 6,444,115, each of which is incorporated herein in relevant part by reference. The reagent layer 128 can include a mediator and an enzyme, and can be deposited onto or affixed to the second electrode 152. Suitable mediators include ferricyanide, ferrocene, ferrocene derivatives, osmium pipyridyl complexes, and quinone derivatives. Suitable enzymes include glucose oxidase, glucose dehydrogenase (GDH) based on pyrroloquinoline quinone (PQQ) co-factor, GDH based on nicotinamide adenine dinucleotide (NAD) co-factor, and FAD-based GDH (EC 1.1.99.10).

Heavy lines in FIG. 2 represent the electrical configuration of the analytical test strip 150. Electric current can flow through the first electrode 151 along a path represented schematically by a conductor 291, and likewise through the second electrode 152, as represented by a conductor 292. For purposes of this description and relating to the system 10, FIG. 1, contacts 293 and 294 are graphic representations of the electrical interfaces between the strip port connector 106, FIG. 1, and the test strip 150. Each such electrical interface can have an impedance resulting from, e.g., oxide formation on the surfaces of electrical contacts. In this example, the contact 293 has an impedance corresponding to the electrical interface between the strip port connector 106 and the second electrical contact pad 212, and the contact 294 corresponds in like manner for the third electrical contact pad 223.

In the example shown, the two electrodes 151, 152 are arranged at least partially in the sample cell 140 to define a capacitor 299 having the received fluid sample as a dielectric. The capacitor 299 in this example additionally has the spacer 235 as a dielectric. However, in various embodiments, the spacer 235 is formed from a plastic having a dielectric constant <4, and the fluid sample is aqueous, with a dielectric constant which is >50 or >80. Therefore, the majority of the capacitance of the capacitor 299 arises from the sample cell 140. When the sample cell 140 does not contain a fluid sample (the test strip 150 is “dry”), the dielectric constant of the sample cell 140 is ˜1.0 (the dielectric constant of air). When the sample cell 140 fills with an aqueous fluid sample (or another fluid sample with a high dielectric constant, e.g., glycerol with a dielectric constant of ˜45), the capacitance of the sample cell 140 increases significantly, with the overall capacitance of the capacitor 299 also increasing significantly.

In addition to serving as a high-constant dielectric, the received fluid sample can provide a resistive path between the two electrodes 151, 152 electrically in parallel with the defined capacitor 299. This is represented graphically by a resistor 298 connecting the conductors 291, 292 in parallel with the capacitor 299. The spacer 235 can contribute some DC resistance (leakage current), but this resistance causes negligible current flow in various embodiments (e.g., DC resistance of the spacer 235 >10 MΩ).

An exemplary test strip was measured using a bench-top capacitance meter. The exemplary strip had a parallel-plate configuration, similar to that shown in FIG. 2. When the test strip was dry, the test strip was measured to have substantially no capacitance between its two electrodes. With a whole blood fluid sample filling the sample cell of the exemplary strip, the test strip had 600-700 nF of capacitance between its electrodes. With a control-solution sample filling the sample chamber, the test strip had 700-800 nF of capacitance between its electrodes.

Once the fluid sample has filled the sample cell 140, the resistor 298 has a real impedance Z_{R}≈R+0j that is substantially a certain number of ohms (R), and is substantially independent of frequency. The capacitor 299 has a complex impedance Z_{C}≈0+−1/ωcj for angular frequency ω (rad/s=2π×f (Hz)) and capacitance C (F). Therefore, as either capacitance or frequency increases, Z_{C} decreases towards 0 and therefore the admittance Y_{C} of the capacitor 299 increases.

The conductors 291, 292 have respective DC resistances, represented graphically as resistors 295, 296. The conductors 291, 292 can also have parasitic capacitances or inductances, as will be apparent to one skilled in the electronics art. Since most of the capacitance between the conductors 291, 292 is located in the sample cell 140, Z_{R} and Z_{C} can be considered as a single parallel element. The network between the contact 293 and the contact 294 can thus be modeled as:

Z_{network}=R₂₉₃+R₂₉₅+Z_{R}∥Z_{C}+R₂₉₆+R₂₉₄ (Eq. 1)

In general, Z_{network} is not a pure resistance because the RC parallel combination has an imaginary component of impedance. However, when an AC signal is applied across this network, as angular frequency ω of the applied signal increases, Z_{C} decreases toward 0. This in turn decreases the parallel-combination term Z_{R}∥Z_{C} toward 0, i.e., the low-impedance capacitor 299 shorts out the resistor 298 when ω is sufficiently high. Above a certain characteristic frequency of the fluid sample in the sample cell 140 of the test strip 150, Z_{R}∥Z_{C}≈0, so the network is effectively:

Z_{network}=R₂₉₃+R₂₉₅+R₂₉₆+R₂₉₄ (Eq. 2)

Measuring the current corresponding to the applied AC voltage thus permits determining Z_{network} as:

$${Z}_{network} = \frac{{V}_{applied , RMS}}{{I}_{meas , RMS}} \qquad ( Eq . 3 )$$

If the contact resistances R₂₉₃ and R₂₉₄ are known, they can be subtracted from Z_{network} to obtain

Rₑ=R₂₉₅+R₂₉₆ (Eq. 4)

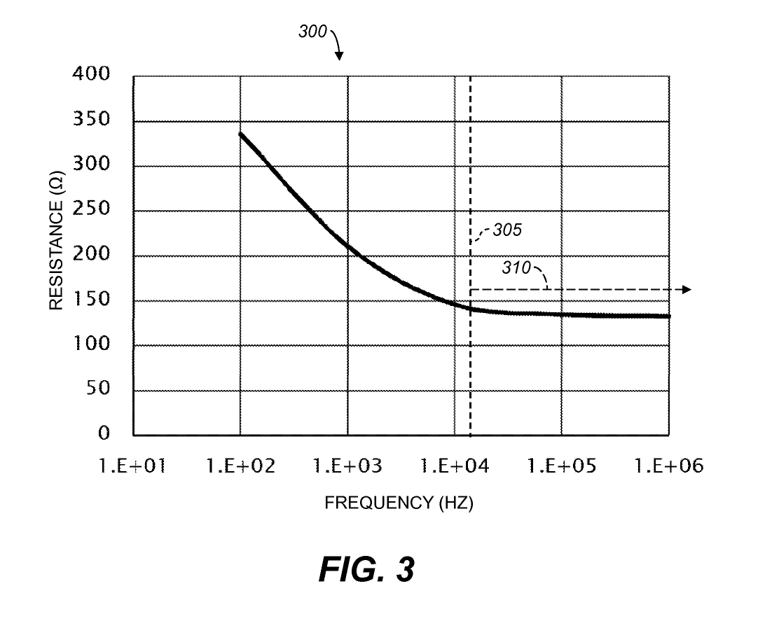

Rₑ can be indicative of the condition of the test strip 150, as will be discussed below with reference to FIG. 3. In general, in various embodiments, the test meter 100, FIG. 1, is further adapted to determine the series resistance Rₑ and indicate to a user that the received analytical test strip 150 is usable if the series resistance Rₑ satisfies a stored resistance criterion, e.g., Rₑ<R_{e,max}.

In Eq. 1, the magnitude of the impedance Z_{C} decreases when either angular frequency ω or capacitance C increases. Therefore, ω and C can be co-optimized. For smaller C, larger ω can be selected, and vice versa. However, as ω increases, the impedance jωL of parasitic inductances L on the test strip 150 increases. It is preferable that C be large enough that such parasitic inductances on the test strip 150 do not contribute significantly to the impedance at the selected ω.

The resultant electrical signal can be a current I_{meas,RMS}, the resistance-characterization circuit 190, FIG. 1, can include a current detector (e.g., a transimpedance amplifier) adapted to provide a measurement voltage Vₘₑₐₛ representative of the current to the microprocessor 186, FIG. 1, and the microprocessor 186 can compute a resistance Rₑ of the analytical test strip 150 using the excitation voltage V_{applied,RMS} and the measurement voltage Vₘₑₐₛ. Vₘₑₐₛ can be, e.g., the product of I_{meas,RMS} and a transimpedance amplifier gain G (Ω).

In various embodiments, impedance can be measured by driving voltage and measuring current, or vice versa. Detectors in the RCC 190 can include potential dividers, peak detectors, or transimpedance amplifiers followed by RMS rectifiers. Measurements can be of a number of ohms, or of simply a pass/fail.

In an example, the fluid sample is a whole blood sample and the excitation frequency (the frequency of V_{applied}) is at least 90 kHz.

In various embodiments, the microprocessor 186 is further configured to determine whether the fluid sample is a bodily-fluid sample or a control sample. This determination can be done by various electrochemical techniques. One example is given in U.S. Pat. No. 8,449,740, incorporated herein by reference in its entirety, in which multiple current transients are measured through an electrochemical test strip. The current transients are then used to determine if a sample is a blood sample or a control solution based on at least two characteristics.

After making the determination, the microprocessor 186 causes the resistance-characterization circuit 190 to provide the excitation voltage signal at a first excitation frequency if the fluid sample is determined to be a bodily-fluid sample and at a second, different excitation frequency if the fluid sample is determined to be a control sample. In various examples, the first excitation frequency (for bodily fluid) is 100 kHz, or at least 90 kHz, and the second excitation frequency (for control solution) is 20 kHz, or at least 15 kHz.

FIG. 3 shows a plot 300 of resistance as a function of frequency. The sample cell in an exemplary test strip was filled with control solution, and impedance measurements were taken at different frequencies using lab-bench equipment. The plot 300 shows the measured resistance, i.e., the magnitude of complex impedance (|Z|); the phase (arg(Z)) is not shown. The phase approached 0° as frequency increased. At and above ˜20 kHz, the strip resistance remains substantially unchanged at −135Ω, regardless of frequency. DC bench measurements were also conducted of the strip electrodes. The 135Ω figure from the AC measurements was determined to correspond to the DC measurements within acceptable tolerances. Specifically, the electrodes were measured to have a resistivity of 8-12 Ω/square (Ω/□), yielding 40Ω resistance along each electrode tested. The contacts were simulated at a 0.5 mm contact size and determined to be 20-30Ω each. Two contacts and two electrodes in series thus are between 120Ω and 140Ω.

The sample cell in an exemplary test strip was filled with blood, and an AC measurement was taken at 100 kHz. The measured resistances was ˜140Ω, which was within normal tolerances. Accordingly, AC measurements can advantageously provide acceptably-accurate results regardless of sample type. Frequencies other than 20 kHz can be used, though preferably the frequency of measurement should be within a stable region 310, e.g., above (or at or above) a characteristic frequency 305 of the fluid sample in the test strip.

The characteristic frequency 305 is a frequency at which the parallel impedance Z_{R}∥Z_{C} is within a selected tolerance of 0+0j. The characteristic frequency 305 depends, e.g., on the geometry of the sample cell 140, FIG. 1, the dielectric constant of the fluid sample, any capacitive contribution from the spacer 235, FIG. 2, and parasitics. Expanding Z_{C} to include these effects, in an example of a conventional parallel-plate capacitor, gives:

$${Z}_{C} \approx 0 + \frac{- 1}{ω \cdot \frac{ɛ A}{d}} j + {Z}_{p} \qquad ( Eq . 5 )$$

for electrodes 151, 152, FIG. 1, facing each other in parallel, with an area A of the sample cell, thickness d of the spacer, dielectric constant 8 of the fluid sample, and parallel capacitive impedance Zₚ. One skilled in the electronics art can adapt this equation, and Eqs. 1-4, according to the specific configuration of a particular test strip. For example, when the electrodes 151, 152 are coplanar rather than facing, Eq. 5 can be modified to take fringing fields into account. Moreover, electrical double layers can develop at the interfaces between the electrodes 151, 152 and the fluid sample. A double layer can include charged particles from the fluid sample adsorbed onto the surface of the electrode, and oppositely-charged particles from the fluid sample electrostatically attracted to the adsorbed particles. These double layers can significantly increase the capacitance. Eq. 1 can be modified to take the double-layer capacitance into account, as is known in the electrochemical and supercapacitor art.

When Eq. 5 is changed, Eq. 1 can be modified accordingly and then used to predict the characteristic frequency 305. For example, an exemplary strip with coplanar, spaced-apart electrodes can have a capacitance in the pF range, as opposed to the nF range for parallel-plate strips.

The results shown in FIG. 3 were determined with AC measurements using a voltage of ˜100 mV RMS. The current consumed during the measurement was simulated, the result of the simulation being ˜740 μA. This current is within the range that can be provided by a portable power supply, such as a coin-cell battery. For a measurement circuit that stabilizes within 10 cycles of the AC excitation, measurement at 100 mV and 740 μA can be performed in 0.1 ms. In an example, the test meter 100 is powered by a CR2032 coin cell battery. Such a battery can provide 3V @ 3 mA continuously, or up to 15 mA for a short duration (e.g., a few milliseconds). If higher measurement currents are desired with such a cell, AC voltage can be increased to draw 10-12 mA for this duration. AAA and various other types of batteries have higher continuous current ratings.

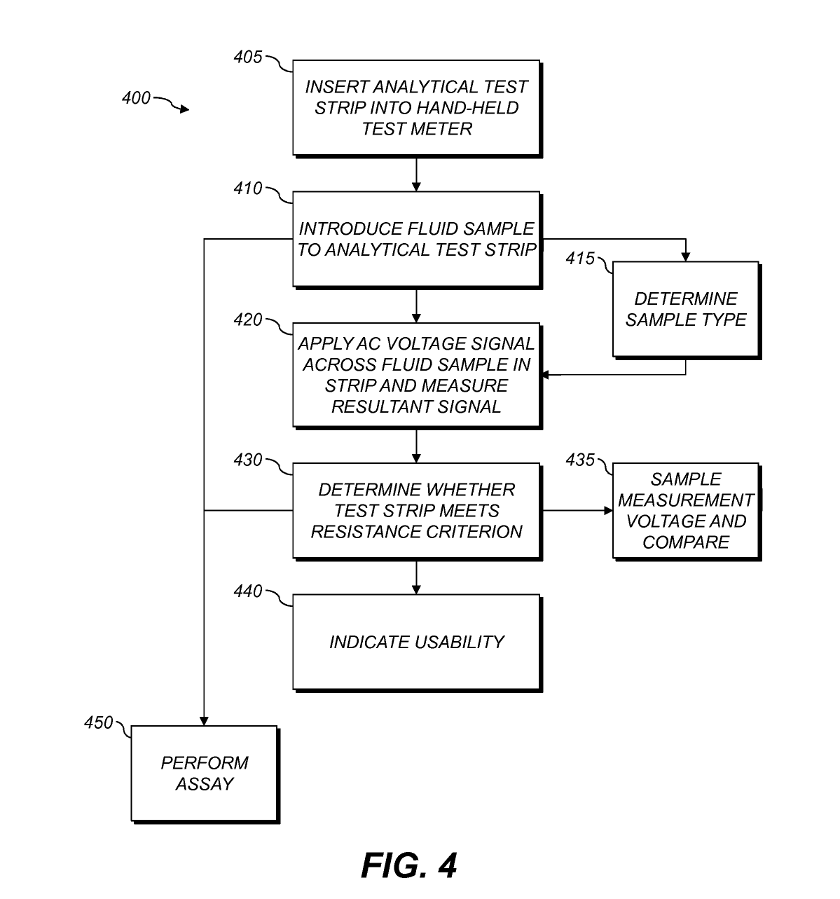

FIG. 4 is a flow diagram depicting stages in a method 400 for operating a hand-held test meter for the determination of usability of an analytical test strip. Reference is made to various components described above for exemplary purposes. Methods described herein are not limited to being performed only by the identified components.

Method 400, at step 405, includes inserting the analytical test strip into the hand-held test meter.

At step 410, a fluid sample, e.g., a whole blood sample, is introduced to the inserted analytical test strip (e.g., into a sample cell of the analytical test strip).

In various embodiments, at step 415, it is determined whether the fluid sample is a bodily-fluid sample or a control sample. A corresponding frequency is selected: a first excitation frequency if the fluid sample is determined to be a bodily-fluid sample, or a second, different excitation frequency if the fluid sample is determined to be a control sample.

At step 420, after the fluid sample is introduced, using the hand-held test meter 100, FIG. 1, an AC voltage signal is automatically applied across the fluid sample in the sample cell. The AC voltage signal has a frequency greater than a characteristic frequency of the fluid sample, e.g., >15 kHz, as discussed above with reference to FIG. 3. In embodiments in which step 415 is used, the AC voltage signal is applied having the selected corresponding frequency. A resultant electrical signal is measured. In at least one embodiment, the AC voltage signal is applied across two electrodes 151, 152 of the analytical test strip 150, each of the two electrodes 151, 152 being disposed at least partly in the sample cell 140.

At step 430, it is automatically determined, e.g., by the microprocessor 186, FIG. 1, whether the analytical test strip 150 meets a selected resistance criterion. This determination is made based on the resultant electrical signal. In an example, the resultant electrical signal is a current and step 430 includes converting the current to a measurement voltage, e.g., using the resistor 192 and the amplifier 193, FIG. 1. Because the electrodes 151, 152 according to this example are disposed at least partly in the sample cell 140, the measurement voltage substantially corresponds to a series resistance of the two electrodes 151, 152.

In various embodiments, the selected resistance criterion is a threshold corresponding to a selected upper resistance limit. At step 430, the resultant electrical signal is compared to the selected resistance criterion. The analytical test strip meets the selected resistance criterion if and only if the resultant electrical signal corresponds to a resistance of the analytical test strip that is less than the selected upper resistance limit.

In various embodiments, step 430 includes step 435. At step 435, the measurement voltage is sampled using an analog-to-digital converter. The sampled measurement voltage is automatically compared to a stored range using the microprocessor 186. The analytical test strip meets the selected resistance criterion if the sampled measurement voltage is within the stored range. The stored range can be open, closed, or semi-open (at either end). For example, the stored range can include voltages corresponding to the range [120Ω,150Ω], or to (−∞,200Ω].

In various embodiments, at step 440, it is indicated to a user of the hand-held test meter that the analytical test strip is usable if the analytical test strip meets the selected resistance criterion. If the test strip fails to meet the selected resistance criterion, it is indicated to the user via the user interface that the analytical test strip is not usable. The indication can be, e.g., a message on the display 181, FIG. 1, an illuminated LED (not shown), a sound or vibration.

In various embodiments, at step 450, an assay is performed, i.e., an analyte is determined in the fluid sample. Step 450 can be performed after step 430 or before step 420.

In various embodiments, a method for operating a hand-held test meter for the determination of usability of an analytical test strip includes the hand-held test meter receiving the analytical test strip having a fluid sample in a sample cell of the inserted analytical test strip. The fluid sample can be introduced before or after the test meter receives the test strip. Using a microprocessor in the test meter, an AC voltage signal is automatically applied across the fluid sample in the sample cell and a resultant electrical signal is automatically measured. The AC voltage signal has a frequency greater than a characteristic frequency of the fluid sample. Using the microprocessor, it is automatically determined whether the analytical test strip meets a selected resistance criterion based on the resultant electrical signal.

Using methods, devices or systems described herein, electrical performance of a strip can be directly measured to determine that there are no significant scratches, film defects or poor contact problems that would significantly compromise an analyte assay performed using that strip. Upper and lower thresholds can set to differentiate usable (good) strips from non-usable (bad) strips. Exemplary strip-good ranges of electrode and contact resistance include 120Ω-150Ω, and <200Ω. Strip-good ranges and thresholds can be set based on manufacturing tolerances of each particular type of test strip. For example, modular strips using miniaturized electrochemical modules (ECMs), can have lower resistances, so the strip-good ranges can include lower values. Ranges and thresholds can be stored in the memory block 118.

In various examples, different types of test strips can be designed to have respective, different resistances, wherein the test meter 100 can store multiple ranges or thresholds for determining the type of strip. The test meter 100 can test the resultant electrical signal (strip resistance) against each range or threshold. For example, strip electrodes can be sputtered for a length of time selected based on the strip type. Resistance can also be changed by adding extra conductive material. In the sample cell 140, FIG. 2, non-reactive material is used (e.g., Au, Pd, C). Outside the sample cell 140, the thicknesses of only certain parts of the electrodes can be determined by masking, or laser-ablate portions of the trace. To increase conductivity, supplemental conductors formed, e.g., from film, copper, or conductive ink, can be added to the strip and electrically connected in parallel with the electrodes.

Some prior testing techniques do not measure resistance directly. Instead, they apply selected DC voltages to test strips and measure peak current magnitudes. Although such techniques are useful, they do not provide direct measurement of the resistance of the test strip electrodes.

Various schemes apply AC waveforms to the fluid sample. These schemes rely on the capacitance or other electrical properties of the fluid sample. In contrast, various embodiments described herein use a high enough frequency ω that the properties of the fluid sample do not significantly affect the result.

Other schemes expressly measure capacitance of the sample cell or the fluid sample therein. Since capacitance is being measured, Z_{C} must be nonzero in these schemes. In contrast, various embodiments herein operate so that Z_{C}≈0. This advantageously improves accuracy of the resistance measurement.

Yet other schemes monitor test strip condition using separate traces to measure resistance. Some schemes use four-wire resistance measurements or modifications thereof, or buffer signals applied to the test strip to compensate for resistance variation. However, these schemes require additional traces or components, increasing size, cost, and complexity of the test strips or the test meter. Moreover, many of these schemes measure across test components that are only representative of the path current takes through the sample cell. Various embodiments described herein advantageously measure resistance along the very same current path taken by measurements, providing increased accuracy.

### PARTS LIST FOR FIGS. 1-4

system
100 test meter
104 housing
106 strip port connector (SPC)
118 memory block
128 reagent layer
140 sample cell
150 analytical test strip
151, 152 electrodes, first, second
180 user interface button
181 display
186 microprocessor
190 resistance-characterization circuit (RCC)
191 AC voltage source
192 resistor
193 amplifier
211, 212 electrical contact pads
215 first electrically-insulating layer
223 electrical contact pad
225 second electrically-insulating layer
227, 228 cutout portions
235 spacer
240 aperture
271 reagent layer
288 proximal end
291, 292 conductors
293, 294 contacts
295, 296 resistors
298 resistor
299 capacitor
300 plot
305 characteristic frequency
310 stable region
400 method
410, 415, 420, 430, 435 steps
440, 450 steps

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. References to “a particular embodiment” and the like refer to features that are present in at least one embodiment of the invention. Separate references to “an embodiment” or “particular embodiments” or the like do not necessarily refer to the same embodiment or embodiments; however, such embodiments are not mutually exclusive, unless so indicated or as are readily apparent to one of skill in the art. The word “or” is used in this disclosure in a non-exclusive sense, unless otherwise explicitly noted. It is intended that the following claims define the scope of the invention and that devices and methods within the scope of these claims and their equivalents be covered thereby.

What is claimed is:

1. A system for determining usability of an analytical test strip, the system comprising:
a) an analytical test strip having two electrodes connected in series with a sample cell, the sample cell being adapted to receive a fluid sample and configured so that the sample cell with the received fluid sample has a characteristic impedance that varies with frequency; and
b) a test meter adapted to receive the analytical test strip, the test meter having at least one contained circuit and a microprocessor, the microprocessor and circuit being configured to detect the presence of the fluid sample in the sample cell of the received analytical test strip and, based upon the detection, cause application of an AC waveform across the sample cell via the electrodes and concurrent measurement of a current through the electrodes, wherein the AC waveform has a frequency at which the characteristic impedance is substantially zero, and the measured current is inversely proportional to a series resistance of the two electrodes.

2. The system according to claim 1, wherein the two electrodes are arranged at least partially in the sample cell to define a capacitor having the received fluid sample as a dielectric, and the received fluid sample provides a resistive path between the two electrodes electrically in parallel with the defined capacitor.

3. The system according to claim 1, wherein the test meter is further adapted to determine the series resistance and indicate to a user that the received analytical test strip is usable if the series resistance satisfies a stored resistance criterion.

4. A hand-held test meter for use with an analytical test strip in the determination of an analyte in a fluid sample, the test meter comprising:
a) a housing;
b) a strip port connector configured to receive the analytical test strip;
c) a resistance-characterization circuit electrically connected to a sample cell of the received analytical test strip via the strip port connector, the circuit being configured to selectively apply an excitation voltage signal to the sample cell to provide a resultant electrical signal, the excitation voltage signal having an excitation voltage and an excitation frequency greater than a characteristic frequency of the fluid sample; and
d) a microprocessor adapted to detect the fluid sample in the sample cell and subsequently cause the resistance-characterization circuit to apply the excitation voltage signal, wherein the microprocessor is further adapted to determine whether the resultant electrical signal satisfies a stored resistance criterion.

5. The test meter of claim 4, wherein the microprocessor is further adapted to indicate to a user via a user interface of the test meter whether the resultant electrical signal satisfies the resistance criterion.

6. The test meter of claim 4, wherein the resultant electrical signal is a current, the resistance-characterization circuit including a current detector adapted to provide a measurement voltage representative of the current to the microprocessor, and the microprocessor being adapted to compute a resistance of the analytical test strip using the excitation voltage and the measurement voltage.

7. The test meter of claim 4, wherein the fluid sample is a whole blood sample and the excitation frequency is at least 20 kHz.

8. The test meter of claim 7, wherein the analytical test strip is an electrochemical-based analytical test strip configured for the determination of glucose in a whole blood sample.

9. The test meter of claim 4, wherein the microprocessor is further configured to determine whether the fluid sample is a bodily-fluid sample or a control sample, and to cause the resistance-characterization circuit to provide the excitation voltage signal at a first excitation frequency if the fluid sample is determined to be a bodily-fluid sample and at a second, different excitation frequency if the fluid sample is determined to be a control sample.

10. The test meter of claim 9, wherein the first excitation frequency is at least 90 kHz and the second excitation frequency is at least 15 kHz.

11. The test meter of claim 4, wherein the strip port connector is configured to operatively interface with the received analytical test strip via a first electrode and a second electrode of the received analytical test strip, the first and second electrodes disposed at least partly in the sample cell of the received analytical test strip.

12. A method for operating a hand-held test meter for the determination of usability of an analytical test strip, the method comprising:
inserting the analytical test strip into the hand-held test meter;
introducing a fluid sample to a sample cell of the inserted analytical test strip;
automatically applying an AC voltage signal across the fluid sample in the sample cell and measuring a resultant electrical signal, wherein the AC voltage signal has a frequency greater than a characteristic frequency of the fluid sample; and
automatically determining whether the analytical test strip meets a selected resistance criterion based on the resultant electrical signal.

13. The method according to claim 12, further including indicating to a user that the analytical test strip is usable if the analytical test strip meets the selected resistance criterion, or else indicating to the user via the user interface that the analytical test strip is not usable if the test strip fails to meet the selected resistance criterion.

14. The method according to claim 12, wherein the resultant electrical signal is a current and the determining step further includes converting the current to a measurement voltage.

15. The method according to claim 14, wherein the determining step includes sampling the measurement voltage using an analog-to-digital converter and automatically comparing the sampled measurement voltage to a stored range using a microprocessor of the hand-held test meter, wherein the analytical test strip meets the selected resistance criterion if the sampled measurement voltage is within the stored range.

16. The method according to claim 14, wherein the applying-signal step includes applying the AC voltage signal across two electrodes of the inserted analytical test strip, each of the two electrodes disposed at least partly in the sample cell, wherein the measurement voltage substantially corresponds to a series resistance of the two electrodes.

17. The method according to claim 14, wherein the applying-signal step includes applying the AC voltage signal via two contacts of the inserted analytical test strip across two respective electrodes of the analytical test strip, each of the electrodes disposed at least partly in the sample cell, wherein the measurement voltage substantially corresponds to a series resistance of the two electrodes and the two contacts.

18. The method according to claim 12, wherein the selected resistance criterion is a threshold corresponding to a selected upper resistance limit and the determining step includes comparing the resultant electrical signal to the selected resistance criterion to determine that the analytical test strip meets the selected resistance criterion if and only if the resultant electrical signal corresponds to a resistance of the analytical test strip that is less than the selected upper resistance limit.

19. The method according to claim 12, further including determining, based on the resultant electrical signal, whether the analytical test strip meets a selected second resistance criterion different from the selected resistance criterion.

20. The method according to claim 12, further including determining whether the fluid sample is a bodily-fluid sample or a control sample, wherein the applying step includes applying the AC voltage signal having a first excitation frequency if the fluid sample is determined to be a bodily-fluid sample and having a second, different excitation frequency if the fluid sample is determined to be a control sample.
